(19) United States
(12) Patent Application Publication
(10) Pub. No.: US 2026/0216491 A1
(43) Pub. Date: Jul. 30, 2026

(54) SYSTEM FOR ATTACHING A HEART VALVE TO A HEART PROSTHESIS AND HEART PROSTHESIS PROVIDED WITH AN ATTACHMENT SYSTEM OF THIS KIND

(71) Applicant: CARMAT, Vélizy-Villacoublay (FR)

(72) Inventors: Sébastien ESTORD, Jouy en Josas (FR); Jean-François PICARD, Carrières sous Poissy (FR)

(21) Appl. No.: 19/148,282

(22) PCT Filed: Sep. 14, 2023
(86) PCT No.: PCT/EP2023/075311

§ 371 Date: Jul. 15, 2025

(30) Foreign Application Priority Data
Jan. 17, 2023 (FR) FR2300413

(51) Int. Cl. A61M 60/122; A61M 60/894; A61M 60/90

(52) U.S. Cl. CPC A61M 60/122; A61M 60/894; A61M 60/90; A61M 2210/125

(57) ABSTRACT

The disclosure relates to a system for attaching a heart valve. The function of the system is to attach a heart valve to a heart prosthesis which is intended to be implanted in the pericardial cavity of a patient and is intended to replace the natural left and right ventricles of the patient, this system comprising a male ring provided with spikes and a cooperating part provided with openings, the male ring or the cooperating part being intended to be secured to the heart prosthesis, the spikes and the openings being configured so that the spikes pass through a collar of the heart valve and enter the openings in an attachment position.

### TECHNICAL FIELD

The present invention relates to a system for attaching a heart valve to a heart prosthesis, said heart prosthesis being implantable in the pericardial cavity of a patient and being capable of replacing the natural left and right ventricles of said patient after their ablation. The present invention also concerns a heart prosthesis provided with such an attachment system.

### PRIOR ART

A fully implantable heart prosthesis, as described for example in the patents FR-2 784 585 and FR-2 902 345, has the function of replacing the ventricles of the patient while retaining the atria. To achieve this, it comprises a rigid prosthesis body wherein artificial ventricles are arranged. To implant this heart prosthesis, i.e. an artificial heart prosthesis, in the pericardial cavity of the patient, it is necessary to create interfaces between the vascular network (atria and arteries) of the patient and the heart prosthesis while respecting the anatomy of the patient.

It is known that the natural heart comprises four heart valves which separate the different cavities and ensure good blood circulation between them, namely two heart valves (mitral valve and tricuspid valve) located between the atria and the ventricles and two heart valves (aortic valve and pulmonary valve) located between the ventricles and the arteries. A fully implantable heart prosthesis, which is intended to replace the natural heart, must therefore incorporate (artificial) heart valves in order to recreate this specific connection with the atria and the arteries.

In order to be able to use conventional heart valves used in valve replacement surgery for this purpose, the fully implantable heart prosthesis must incorporate a specific interface. A standard heart valve generally consists of three leaflets that open and close under the action of pressure, three legs to which the leaflets are attached and a base that allows the surgeon to suture the valve. The base may be flat or corrugated (in the shape of a wave).

Such heart valves may be used for the intake or the ejection of blood into the heart prosthesis.

Generally, for the ejection, the heart valve is of the flat-base type and rests with its flat base in a channel of the heart prosthesis, the legs being oriented towards the outside of the ventricles. A fabric duct is arranged in the channel of the heart prosthesis above the heart valve. A locking system secures the assembly to the heart prosthesis, and the fabric duct is then sutured to the artery of the patient by the surgeon.

This attachment mode used at the ejection is not completely satisfactory and has the following disadvantages:
a short distance between the highest point of the heart valve and the point where the tissue duct is sutured to the artery (pulmonary artery or aorta) of the patient, which may hamper the surgeon's ability to perform the suture;
the need to compress the annulus of the heart valve and the tissue duct in the channel of the heart prosthesis to ensure the blood-tightness of the connection, which requires a significant insertion effort on the part of the surgeon. This operation is made even more complex by the fact that the heart prosthesis does not have a gripping point and there is a risk of twisting the locking clip;
the materials and the dimensions of the assembly formed by the heart valve and the tissue duct, which is installed in the channel of the heart prosthesis, are not the best in terms of tightness; and
this attachment method does not allow the use of heart valves with a non-flat base, for example in the shape of a wave.

### SUMMARY OF THE INVENTION

The object of the present invention is to remedy at least some of the aforementioned disadvantages, with the aim of simplifying and improving the operation of attaching artificial heart valves to a heart prosthesis (intended to be implanted in the pericardial cavity of a patient in order to replace the natural left and right ventricles of the patient), and to be able to use heart valves of various designs and in particular heart valves with non-flat bases. To this end, it relates to an attachment system intended for attaching a (artificial) heart valve to a heart prosthesis.

According to the invention, said attachment system comprises a male ring provided with spikes, and a cooperating part provided with openings, said male ring or said cooperating part being intended to be secured to the heart prosthesis, and in that the spikes and the openings are configured so that said spikes pass through a collar of the heart valve and enter said openings in a so-called attachment position.

Thus, thanks to the invention, the male ring and the cooperating part which are intended to hold the heart valve at the level of the collar, may be adapted to this collar, namely to the base of the heart valve, as specified below. The attachment system may thus be adapted to heart valves of varicose construction and in particular heart valves with non-flat bases, both for heart valves used for the ejection and for heart valves used for the intake.

As also specified below, said attachment system has many other advantages, including the following advantages:
it does not lengthen operating time in the operating theatre (for example, no suturing with the interface parts);
it may be easily mounted by the surgeon;
it ensures that there is no leakage of blood from the inside of the ventricles to the outside of the heart prosthesis;
it allows the heart valve to be kept in its packaging and opened for the implantation only in the operating theatre; and
it allows the sinus of Valsalva to be retained for the intake portion of the heart prosthesis.

Depending on the embodiment envisaged, and as specified below, one of the two elements (the male ring or the cooperating part) is intended to be made secured to the heart prosthesis.

Advantageously, the male ring and the cooperating part each have a so-called contact face, these contact faces being intended to grip at least the collar of the heart valve in the attachment position, and in that said contact faces have shapes adapted to the shape of the collar of the heart valve.

In a first embodiment, the spikes and the openings are configured so that the spikes are substantially straight in the attachment position.

Furthermore, in a second embodiment, the spikes and the openings are configured so that the spikes are bent (or curved) in the attachment position, for example angled or with a curved shape. This deformation strengthens the holding of the spikes in the openings and thus the holding between the various parts connected together.

The spikes may have a constant diameter. However, in a particular embodiment, the spikes have a diameter which reduces towards a free end, which makes it easier to hold the spikes in the openings.

In a preferred embodiment, the male ring, which comprises at least one annular support and the spikes secured to the annular support, is a one-piece part, which makes it easier to manufacture. In an alternative embodiment, the spikes may be fitted to the annular support.

In a first embodiment, the cooperating part is a female ring. In this first embodiment, depending on the embodiment alternative envisaged, one of these two rings (the male ring or the female ring) is intended to be attached to the heart prosthesis.

Advantageously, the female ring comprises a face opposite the contact face, which has a shape complementary to a shape of a so-called receiving face of the heart prosthesis.

In a second embodiment, the cooperating part corresponds to a portion of the heart prosthesis. Preferably, said portion of the heart prosthesis comprises a tubular element which is provided, on its internal periphery, with a rim intended to receive the collar of the heart valve.

Furthermore, in a particular embodiment, for a heart valve intended for the ejection, the spikes and the openings are configured so that said spikes also pass through a collar of a duct (intended to be sutured to an artery) in the attachment position.

In addition, in a particular embodiment, the attachment system further comprises, at least one of the following elements:
an O-ring;
an attachment clip;
a holding ring.

The present invention also relates to a heart prosthesis implantable in the pericardial cavity of a patient, said heart prosthesis being adapted of replacing the natural left and right ventricles of said patient and comprising at least one heart valve.

According to the invention, the heart prosthesis comprises at least one attachment system such as that described above, for attaching the heart valve to the heart prosthesis.

In a preferred embodiment, the heart prosthesis comprises two, three or (preferably) four heart valves, each of which is attached to the heart prosthesis by means of an attachment system such as that described above.

### BRIEF DESCRIPTION OF THE FIGURES

The appended figures make it clear how the invention may be carried out. In these figures, identical references designate similar elements.

FIG. 1 is an exploded perspective view of a first embodiment of an attachment system.

FIG. 2 is a perspective view of the attachment system of [FIG. 1], in a mounted position.

FIG. 3 is a perspective view of the attachment system of [FIG. 1], in an attachment position.

FIG. 4 is a sectional view of the attachment system in the attachment position of [FIG. 3].

FIG. 5 is an exploded perspective view of a first variant of a second embodiment of an attachment system.

FIG. 6 is a sectional view of the attachment system of [FIG. 5], in an attachment position.

FIG. 7 is an exploded perspective view of a second variant of a second embodiment of an attachment system.

### DETAILED DESCRIPTION

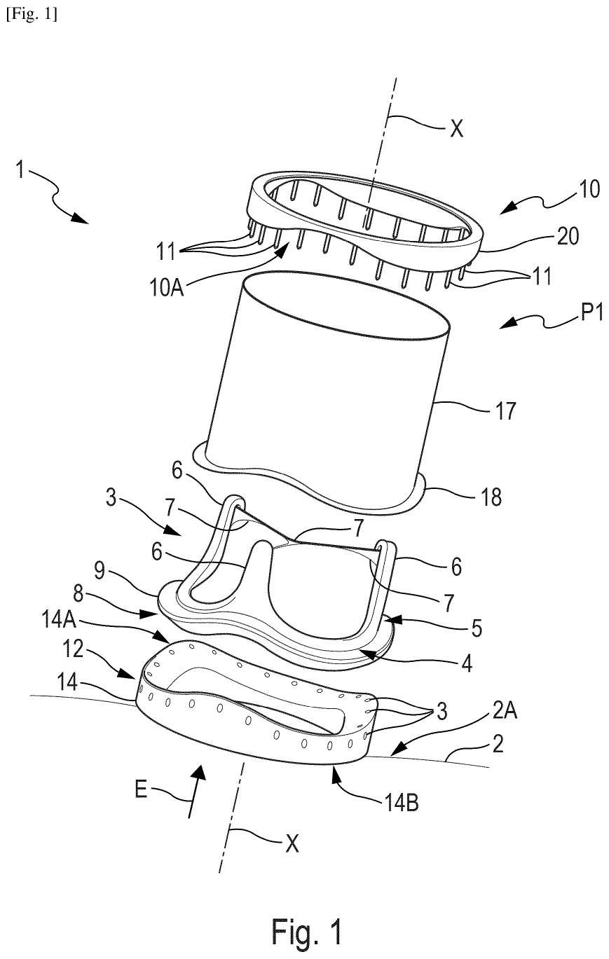

The attachment system 1 illustrating the invention and schematically represented in various specific embodiments in FIGS. 1 to 7 has the function of attaching an artificial heart valve (intended to replace a natural heart valve of the patient) to a heart prosthesis.

This heart prosthesis 2 (shown very partially and very schematically in FIGS. 1 and 5 to 7) may be implanted in the thoracic and pericardial cavity of a patient and is suitable for replacing the natural left and right ventricles of the patient after their ablation. To achieve this, the heart prosthesis 2 comprises a rigid prosthesis body (not shown), wherein artificial left and right ventricles are arranged to replace the natural left and right ventricles of the patient.

The heart prosthesis 2 comprises all the means necessary for its operation. In particular, it may comprise at least some of the characteristics presented in the aforementioned patents FR-2 784 585 and FR-2 902 345. The heart prosthesis 2, and in particular the elements arranged in the prosthesis body, are not described further in the following description.

The heart prosthesis 2 intended to replace the natural heart of the patient comprises, like the natural heart, four heart valves, namely two heart valves (mitral valve and tricuspid valve) provided between the atria and the artificial ventricles of the heart prosthesis 2, for the intake, and two heart valves (aortic valve and pulmonary valve) provided between the artificial ventricles of the heart prosthesis 2 and the arteries (aorta and pulmonary artery) of the patient, for the ejection.

In a preferred implementation, at least one heart valve 3, but preferably all four heart valves 3, each correspond to a conventional heart valve used in the valve replacement surgery.

In a particular embodiment, each (artificial) heart valve 3 comprises, as shown in [FIG. 1], an annular-shaped base 4, which is provided, on a downstream face 5, with three legs 6. The heart valve 3 also comprises three leaflets 7. Each leaflet 7 is attached to two adjacent legs 6 and to the base 4. The three leaflets 7 of the heart valve 3 are configured to open and close under the action of pressure in the upstream portion. In this description, the terms upstream and downstream are defined according to the direction of blood flow through the heart valve 3, as illustrated by arrow E in FIGS. 1, 2, 5 and 7.

In addition, the base 4 has an upstream face 8, opposite the downstream face 5, and comprises a collar 9 which surrounds it (around its entire outer periphery). The upstream face 8 (and the collar 9) may have a variety of shapes. In a first embodiment (not shown), the upstream face 8 and the collar 9 are flat. In a second particular embodiment, as shown in particular in FIGS. 1 and 5, the upstream face 8 and the collar 9 are curved or corrugated, for example in the general shape of a wave.

The function of the attachment system 1 is to attach such a heart valve 3, whatever the shape of its base 4, to the heart prosthesis 2.

To do this, the attachment system 1 comprises, as shown in particular in FIG. 1, an annular male ring 10, which is provided on one face 10A (longitudinal) with spikes 11. The spikes 11 correspond to elongated elements (in particular solid tubes) projecting from the face 10A.

In a preferred embodiment, the male ring 10 (which comprises in particular an annular support 20 and the spikes 11 secured to the annular support 20) is a one-piece part, which makes it easier to manufacture. This one-piece part is made of titanium, for example. It is also possible, in a variant of embodiment not shown, for the spikes to represent individual elements which are fitted (for example welded or glued) to the annular support.

The attachment system 1 also comprises a cooperating part 12 intended to cooperate with the male ring 10. This cooperating part 12 is provided with openings 13. Each of the openings 13 is intended to receive a spike 11 of the male ring 10 in an attachment position PF (FIGS. 3, 4 and 6). The cooperating part 12 thus comprises at least as many openings 13 as the male ring 10 comprises spikes 11, and the spikes 11 and the openings 13 are shaped and arranged so that each of the spikes 11 may be inserted into an opening 13.

For the purposes of the present invention, the attachment position PF is taken to represent the position wherein the heart valve 3 is integrated into the heart prosthesis 2 and attached, in its final position, to the heart prosthesis 2 by means of the attachment system 1.

One of the following elements: the male ring 10, the cooperating part 12, is intended to be secured to the heart prosthesis 2. This connection may be achieved in a number of different embodiments, as described below.

In addition, the spikes 11 and the openings 13 are configured, as also specified below, in such a way that the spikes 11 pass through the collar 9 (formed, for example, from a strip of silicone and a porous PTFE fabric) of the heart valve 3, before entering the openings 13, in the attachment position PF.

Consequently, in the attachment position PF, the male ring 10 and the cooperating part 12 grip the collar 9 of the heart valve 3, as shown in FIGS. 3, 4 and 6.

In addition, as explained below, the spikes 11 are held by lateral contact in the openings 13, which allows to secure the male ring 10 to the cooperating part 12 and hold them together, possibly (in one particular embodiment) with the aid of an additional element (for example a clip). This connection allows the heart valve 3 to be held in place by means of the collar 9 and thus the heart valve 3 to be attached to the cooperating part 12 and, consequently, to the heart prosthesis 2 (to which the cooperating part 12 is secured).

The male ring 10 and the cooperating part 12 each have a so-called contact face 10A and 14A ([FIG. 1]). The contact faces 10A and 14A are intended to grip the collar 9 in the attachment position PF. These contact faces 10A and 14A have shapes adapted to the shape of the collar 9 and therefore to the shape of the base 4.

The attachment system 1 may thus be adapted to the shape of the collar 9 and of the base 4 of the heart valve 3. In particular, it may be adapted to a heart valve 3 with a corrugated base 4. In this way, the attachment system 1 may be used to attach any type of conventional heart valve 3, and in particular a heart valve 3 with a non-flat base 4, for its integration into the heart prosthesis 2.

In addition, the attachment system 1 may be used either to attach a heart valve 3 intended for ejecting blood out of the heart prosthesis 2, or to attach a heart valve 3 intended to the intake of blood into the heart prosthesis 2, as specified above.

The attachment system 1, as described in more detail below, represents an interface which allows to satisfy the following constraints associated with its use in an implantable heart prosthesis 2:
it is biocompatible;
it may be implanted for a long time;
it may be cleaned and suitable to be sterilized;
it guarantees the sealing with the heart prosthesis 2; and
it is provided with an ergonomic and rapid attachment mode (which allows to reduce the duration of the extracorporeal circulation).

The attachment system 1 may be produced in different ways. In particular, it may be produced in two different ways, depending on how the cooperating part 12 or the male ring 10 is secured to the heart prosthesis 2.

In a first embodiment shown in FIGS. 1 to 4, the cooperating part 12 is a female ring 14, i.e. an independent part intended to be connected to the heart prosthesis 2. The female ring 14 is a counter-form of the male ring 10. In this first embodiment, one of the two rings (the male ring 10 or the female ring 14) is attached to the heart prosthesis 2.

Thus, in a first (preferred) variant, the female ring 14 is attached in the usual way to the heart prosthesis 2, and in a second variant, the male ring 10 is attached in the usual way to the heart prosthesis 2.

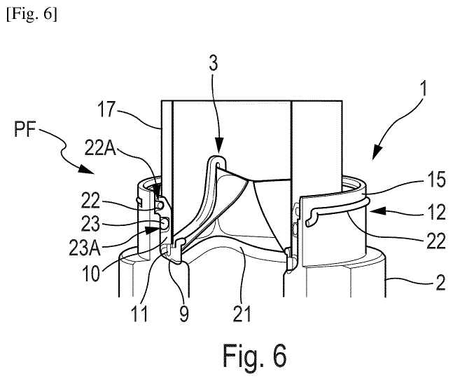

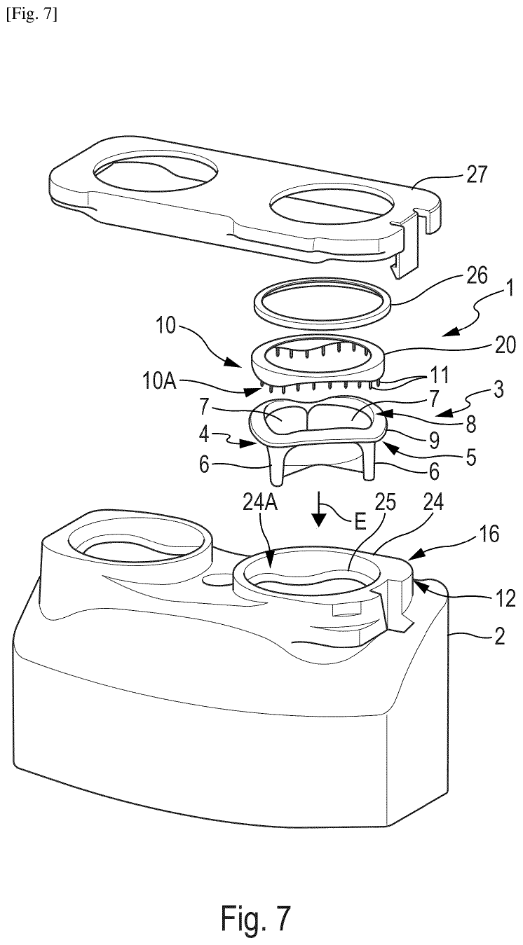

Furthermore, in a second embodiment, the cooperating part 12 corresponds to a portion 15, 16 of the heart prosthesis 2, as represented according to two different variants in FIGS. 5 to 7, namely a first variant in FIGS. 5 and 6 for the attachment of a heart valve 3 intended for the ejection and a second variant in [FIG. 7] for the attachment of a heart valve 3 intended for the intake.

In the first embodiment shown in FIGS. 1 to 4, the cooperating part 12 is therefore a female ring 14, for example made of titanium, initially independent and separate from the heart prosthesis 2, which has an upstream face 14B of particular shape, intended to come into contact with the heart prosthesis 2. This particular shape is complementary to the shape of a downstream face 2A known as the receiving face (shown very schematically in [FIG. 1]) of the heart prosthesis 2.

This first embodiment comprising the female ring 14, shown in FIGS. 1 to 4, relates to an attachment system 1 for attaching a heart valve 3 intended for the ejection.

FIG. 1 shows the various elements in an exploded view. In this embodiment (therefore intended for the ejection), the function of the attachment system 1 is to connect a duct 17 to the heart prosthesis 2, in addition to the heart valve 3. This duct 17 is intended to carry blood ejected from the heart prosthesis 2 (in the direction of arrow E) through the heart valve 3 to an artery (aorta or pulmonary artery) to which the duct 17 is sutured in the usual way.

The duct 17 made of fabric also comprises a collar 18 at its upstream end. This collar 18, made from polyester for example, is similar in shape to the collar 9 on the heart valve 3. In this particular embodiment, the spikes 11 of the male ring 10 are intended to also pass through (or pierce) the collar 18 of the duct 17.

In order to be passed through or pierced by the spikes 11, the collar 9 of the heart valve 3, and where applicable, the collar 18 of the duct 17 must either comprise holes (of appropriate size), or (preferably) be made of a material allowing the spikes 11 to pierce them.

FIGS. 1, 2 and 3 show three successive steps in the attachment of the heart valve 3 and the duct 17 to the heart prosthesis 2.

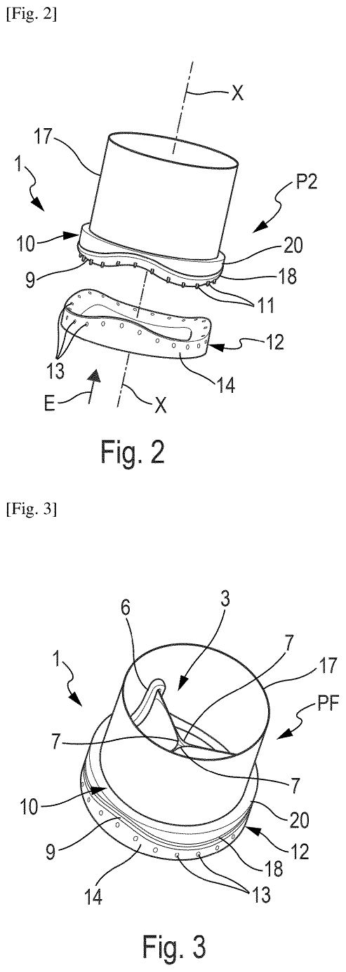

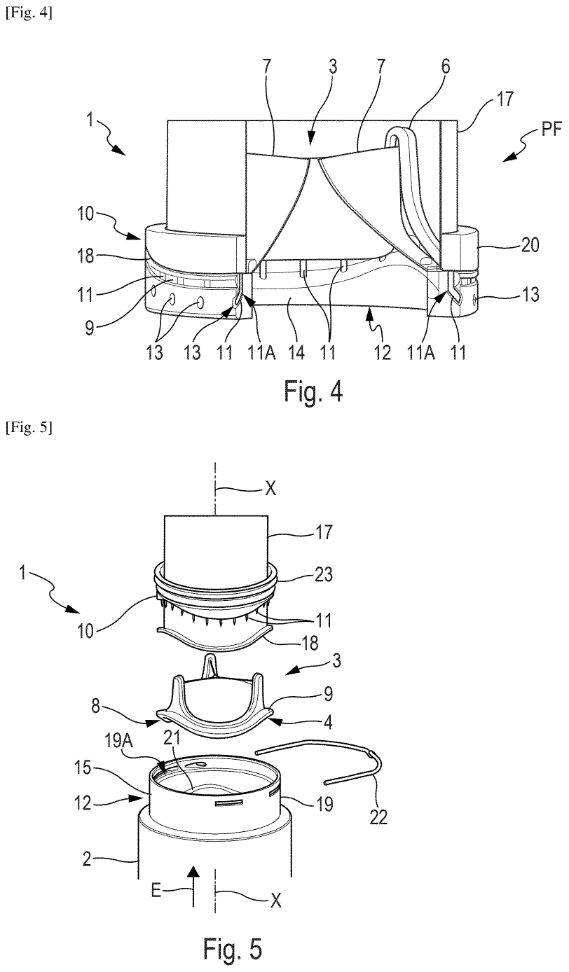

More precisely:
[FIG. 1] illustrates an initial position P1 wherein the various elements are not connected together (or assembled);
[FIG. 2] illustrates an intermediate position P2 during the assembly, wherein the male ring 10 has been assembled to the collar 18 of the duct 17 and to the collar 9 of the heart valve 3. The spikes 11 of the ring 10 pass through these collars 9 and 18; and
FIG. 3 shows the attachment position PF. This attachment position PF is also shown (in cross-section) in [FIG. 4].

In this embodiment, the spikes 11 of the male ring 10 therefore pierce the collar 18 of the duct 17 and the collar 9 of the heart valve 3 before being inserted into the openings 13 of the female ring 14.

In a preferred embodiment, the spikes 11 of the male ring 10 are arranged parallel to the longitudinal axis X-X of the heart valve 3 and of the duct 17 when the male ring 10 is positioned coaxially with respect to this longitudinal axis, X-X, as shown in FIGS. 1 to 7.

In the first embodiment, the female ring 14 comprises openings 13 of cylindrical shape, i.e. rectilinear, which are inclined with respect to the longitudinal axis X-X, i.e. their orientation presents a non-zero angle with respect to this longitudinal axis X-X as shown in [FIG. 4],

Thus, subjected to a pressure during the assembly and constrained by the inclination of the openings 13, the spikes 11 are caused to bend (or curve), as shown in [FIG. 4]. Consequently, on entering the openings 13, the spikes 11 change direction and present a bend 11A ([FIG. 4]) in their attachment position PF. In a variant embodiment (not shown), they may have another curved shape, different from this shape with a bend.

This mechanical deformation allows the assembly of the four parts (male ring 10, collar 18 of the ejection duct 17, collar 9 of the heart valve 3 and female ring 14) to become a one-piece assembly.

The female ring 14 may be adapted to the content and the shape of the heart prosthesis 2 and thus facilitates the integration of the heart valve 3. The male ring 10 and the female ring 14 may be adapted to allow, regardless of the embodiment considered, any type of heart valve 3 to be attached without having to modify the design of the heart prosthesis 2.

The attachment system 1 may be produced in different sizes and/or shapes in order, in particular, to adapt to various surgical heart valves 3.

In the first embodiment, only a (preferred) embodiment intended for the ejection is shown. It is also possible to provide such an embodiment with cooperating male and female rings for the intake.

In addition, in order to adapt as closely as possible to the heart prosthesis 2 and to minimize the overall dimensions of the attachment system 1, the second embodiment makes provision for eliminating the female ring by adapting the shape of a portion of the heart prosthesis 2 to a counter-shape of the male ring 10.

In this second embodiment, the cooperating part 12 therefore corresponds to a portion 15, 16 of the heart prosthesis 2. This second embodiment may also be used to attach a heart valve 3 intended for the ejection or for the attachment of a heart valve 3 intended for the intake.

In the first variant of this second embodiment, shown in FIGS. 5 and 6, which is also intended, like the first embodiment of FIGS. 1 to 4, to attach a heart valve 3 used for the ejection, the cooperating part 12 corresponds to a portion 15 of the heart prosthesis 2. This portion 15 of the heart prosthesis 2 comprises, in this example, a tubular element (or stretch) 19.

In this embodiment, the tubular element 19 is provided all around its internal periphery 19A with a rim 21 of a shape adapted to the shape of the base 4 of the heart valve 3. The wave shape of the heart valve 3 is thus integrated into the body of the heart prosthesis 2. The upstream face 8 of the base 4 of the heart valve 3 rests on this rim 21 in the attachment position PF, as shown in [FIG. 6].

In this variant of FIGS. 5 and 6, the function of the spikes 11 of the male ring 10 is still to pierce the collar 18 of the duct 17 and the collar 9 of the heart valve 3, before being housed in the cooperating part 1, previously machined in the body of the heart prosthesis 2.

In this particular embodiment, the openings 13 are parallel to the longitudinal axis X-X of the attachment system 1, so that, in the attachment position PF, the spikes 11 are not curved (or bent) but remain straight and parallel to the longitudinal axis X-X, as seen in [FIG. 6].

In this embodiment, to increase the holding of the male ring 10, a clip 22 may be provided, which is produced in the usual way to hold the assembled parts together. For this purpose, a conventional groove 22A ([FIG. 6]) is preferably provided, intended to receive the clip 22.

In addition, in the example shown in FIGS. 5 and 6, an O-ring 23 is also provided in a groove 23A ([FIG. 6]) of the male ring 10, which is intended to seal the assembled parts with respect to the blood flow.

In the context of the present invention, whatever the embodiment, the spikes 11 may have a constant diameter. However, in a particular embodiment, the spikes 11 have a diameter which reduces towards their free end (i.e. their end opposite that which is connected to the face 10A of the male ring 10), which facilitates the mounting and strengthens the hold, when they are inserted in cylindrical openings 13 (i.e. of constant diameter).

This first variant of the second embodiment comprising the portion 15 as a counter-form of the male ring 10 allows a good adaptation to the heart prosthesis and minimizes the overall dimensions of the attachment system 1.

This variant, which requires the design of the heart prosthesis 2 to be modified if the shape (for example) of the heart valve 3 changes, nevertheless offers the advantage of reducing the projection height of the legs of the heart valve 3.

In addition, the second variant of the second embodiment, shown in [FIG. 7], is intended to attach a heart valve 3 used for the intake. In this case, the free ends of the leaflets 7 of the heart valve 3 are directed towards the heart prosthesis 2, i.e. in the opposite direction to the embodiment shown in FIGS. 5 and 6.

In this second variant, the cooperating part 12 corresponds to a portion 16 of the heart prosthesis 2. This portion 16 of the heart prosthesis 2 comprises, in this example, a tubular element (or stretch) 24.

In this embodiment, the tubular element 24 is provided all around its internal periphery 24A with a rim 25 of a shape adapted to the shape of the base 4 of the heart valve 3. The wave shape of the heart valve 3 is thus integrated into the body of the heart prosthesis 2. The upstream face 8 of the base 4 of the heart valve 3 rests on this rim 25 in the attachment position (not shown).

In addition, the male ring 10, which is in contact with blood, is made hemocompatibility.

In this embodiment, intended for the intake, a holding ring 26, preferably a metal annulus, is used to hold the male ring 10 with the spikes 11 and the heart valve 3 in the heart prosthesis 2 until it is installed on a conventional metal bezel 27, already in place in the thorax of the patient, when the heart prosthesis 2 is implanted. By way of illustration, the patents FR-2 902 343 and FR-2 902 344 describe a system of cooperating bezels the intakes of the heart prosthesis.

In an alternative embodiment (not shown), the annular support 20 of the male ring 10 may be sized and shaped to provide this holding (before installation on the metal bezel), instead of the holding ring 26.

It is also possible to provide, for the intake, an attachment system according to the first embodiment with cooperating male and female rings. In such an embodiment (not shown), the portion 16 of the example in [FIG. 7] is replaced by a female ring intended to be attached to the heart prosthesis.

Furthermore, in the context of the present invention, depending on the particular embodiment considered, the attachment system 1 may comprise one or more of the following elements:
an O-ring such as, for example, the O-ring 23 shown in FIGS. 5 and 6;
an attachment clip such as, for example, the clip 22 shown in FIGS. 5 and 6; and
a holding ring such as, for example, the holding ring 26 shown in [FIG. 7],

In addition, whatever the embodiment considered, to meet the hemocompatibility constraint, the part or parts of the attachment system 1 in contact with blood (mainly the female ring 14 in the embodiment shown in FIGS. 1 to 4) may be covered with a knitted prosthesis (polyester yarn knitting weave). Preferably, in application to the female ring, this polyester fabric is cut as close as possible to the shape of the ring, wrapped around the ring and sutured to hold it in place.

Alternatively, to make the parts in contact with blood hemocompatibility, other solutions may be provided such as, for example, covering the surface in contact with blood with cPTFE, titanium bead blasting, etc.

Regardless of the embodiment considered, the method of installing the attachment system 1 comprises at least one operation consisting of piercing the collar 9 of the heart valve 3 (and, where appropriate, the collar 18 of the duct 17) by the spikes 11 of the male ring 10 and inserting said spikes 11 into the cooperating openings 13 of the cooperating part 12. Depending on the embodiment, the method comprises other steps implemented before and after this operation.

The attachment system 1, as described above, replaces suture operations, while providing a rigid armature for the heart valve 3, which facilitates its integration into the design of the heart prosthesis 2.

The attachment system 1 has many advantages. Firstly, it offers the following key benefits:
reducing the distance between the highest point of the heart valve 3 and the highest point of the interface at the level of the ejection duct of the heart prosthesis 2;
allowing the use of a heart valve 3 whose base 4 is not flat but incorporates a wave shape;
simplifying the preparation and the implantation of the heart prosthesis 2.

In addition, the attachment system 1 also has the following advantages:
it does not lengthen operating time in the operating theatre (for example, no suturing with the interface parts);
it may be easily mounted by the surgeon;
it ensures that there is no leakage of blood from the inside of the ventricles to the outside of the heart prosthesis 2;
it allows the heart valve 3 to be kept in its packaging and opened for implantation only in the operating theatre; and
it allows the sinus of Valsalva to be retained for the intake portion of the heart prosthesis 2.

1. An attachment system intended for attaching a heart valve to a heart prosthesis, said heart prosthesis being intended to be implanted in a pericardial cavity of a patient and to replace natural left and right ventricles of the patient, wherein the attachment system comprises a male ring provided with spikes, and a cooperating part provided with openings, said male ring or said cooperating part being intended to be secured to the heart prosthesis, and in that the spikes and the openings are configured so that said spikes pass through a collar of the heart valve and enter said openings in a so-called attachment position.

2. The attachment system according to claim 1, wherein the male ring and the cooperating part each have a so-called contact face, these contact faces being intended to grip at least the collar of the heart valve in the attachment position, and in that said contact faces have shapes adapted to the shape of the collar of the heart valve.

3. The attachment system according to claim 1,
wherein the spikes and the openings are configured so that the spikes are substantially straight in the attachment position.

4. The attachment system according to claim 1,
wherein the spikes and the openings are configured so that the spikes are bent in the attachment position.

5. The attachment system according to claim 1,
wherein the spikes have a diameter which reduces towards a free end.

6. The attachment system according to claim 1,
wherein the male ring comprises at least one annular support and the spikes secured to the annular support is a one-piece part.

7. The attachment system according to claim 1,
wherein the cooperating part is a female ring.

8. The attachment system according to claim 7, wherein the female ring comprises a face, opposite the contact face, which has a shape complementary to a shape of a so-called receiving face of the heart prosthesis.

9. The attachment system according to claim 1,
wherein the cooperating part corresponds to a portion of the heart prosthesis (2).

10. The attachment system according to claim 9, wherein said portion of the heart prosthesis comprises a tubular element which is provided on its internal periphery with a rim intended to receive the collar of the heart valve.

11. The attachment system according to claim 1,
wherein, for a heart valve intended for an ejection, the spikes and the openings are configured so that said spikes also pass through a collar of a duct in the attachment position.

12. The attachment system according to claim 1,
further comprising at least one of the following elements:
an O-ring;
an attachment clip; and
a holding ring.

13. A heart prosthesis implantable in the pericardial cavity of a patient, said heart prosthesis being capable of replacing the natural left and right ventricles of said patient and comprising at least one heart valve,
further comprising at least one attachment system according to claim 1 for attaching the heart valve to the heart prosthesis.

14. The heart prosthesis as claimed in claim 13, further comprising two, three or four heart valves, each of which is attached to the heart prosthesis by means of the attachment system.

15. The system according to claim 2,
wherein the spikes and the openings are configured so that the spikes are substantially straight in the attachment position.

16. The system according to claim 2,
wherein the spikes and the openings are configured so that the spikes are bent in the attachment position.

17. The system according to claim 2,
wherein a female ring comprises a face, opposite the contact face, which has a shape complementary to a shape of a so-called receiving face of the heart prosthesis.
